(19) United States
(12) Patent Application Publication
(10) Pub. No.: US 2016/0258936 A1
(43) Pub. Date: Sep. 8, 2016

(54) METHOD FOR ANALYZING GAS SAMPLES

(71) Applicant: TURUN YLIOPISTO, Turun yliopisto (FI)

(72) Inventors: Harri HÄRMMÄ, Turku (FI); Pekka HÄNNINEN, Turku (FI)

(73) Assignee: TURUN YLIOPISTO, Turun yliopisto (FI)

(21) Appl. No.: 15/031,455

(22) PCT Filed: Oct. 23, 2014
(86) PCT No.: PCT/FI2014/050805

§ 371 Date: Apr. 22, 2016

(30) Foreign Application Priority Data
Oct. 23, 2013 (FI) 20130307

(51) Int. Cl. G01N 33/52

(52) U.S. Cl. CPC G01N 33/52

(57) ABSTRACT

This invention relates to a method for analyzing gas samples, particularly with the aid of lanthanide(III) ions and ligands. The invention relates also to a kit for analyzing gas samples utilizing the properties of lanthanide(III) chelates.

### FIELD OF THE INVENTION

This invention relates to a method of analyzing gas samples, particularly with the aid of lanthanide(III) ions and ligands. The invention relates also to a kit for analyzing gas samples utilizing the properties of lanthanide(III) chelates.

### BACKGROUND OF THE INVENTION

The publications and other materials used herein to illuminate the background of the invention, and in particular, cases to provide additional details respecting the practice, are incorporated by reference.

Current devices for exhaled breath diagnostics are based on the detection of volatile organic compounds (VOC) in the gas phase. Often mass spectrometry or a variant thereof is used to distinguish the different VOC species.

Fu et al. (Cancer Medicine 2014:3,174) demonstrate a quantitative analysis of carbonyl volatile organic compounds and identification of lung cancer in exhaled breath using silicon microreactor technology consisting of thousands of micropillars coated with an ammonium aminooxy salt for capture of carbonyl VOCs by means of oximation reactions. Captured aminooxy-VOC adducts are analyzed by nanoelectrospray Fourier transform-ion cyclotron resonance mass spectrometry. The carbonyl VOC profile in exhaled breath determined using the silicon microreactor technology provides for the noninvasive detection of lung cancer.

A Proton Transfer Reaction-Mass Spectrometry (PTR-MS) technology enables real-time measurement of volatile organic compounds. The fundamental process in the PTR-MS is that protonated water (H₃O⁺) interacts with the trace gas (R). During this interaction, a proton transfers from the hydronium to the trace gas molecule, which leads to a protonated and therefore ionized molecule (RH⁺) and a neutral water molecule (H₂O). The detection of the ionized molecules is based on quadrupole or time of flight mass spectrometer.

A BreathSpec technology includes a spirometer for reliable breath volume sampling. The method uses Ion Mobility Spectrometry as an analytical technology to separately detect gaseous compounds in a mixture of analytes. The separation is based on the specific drift times that ionized compounds need to pass a drift tube with fixed distance in a defined electric field. The drift time of each substance is determined using mass spectrometry.

Another system suitable for use in gas analysis captures a precise amount of gas or breath and detects the volatile organic compounds by means of colorimetric sensor array (CSA). The CSA is a matrix of colored chemical indicators of diverse reactivities embedded in a nanoporous sol-gel matrix. Each indicator has distinct chemical reactivity with volatile species and changes color differently upon exposure to analytes. Optical sensors are used to detect the color changes.

In electronic nose approaches, gas molecules interact with solid-state sensors by absorption, adsorption, or chemical reactions with thin or thick films of the sensor material. The sensor device detects the physical and/or chemical changes incurred by these processes and these changes are measured as an electrical signal. A number of electronic nose approaches have been developed.

Despite of a number of different technologies and systems available, there is a need in the art for a simple, cost-efficient and reliable technology for analyzing gas samples, such as respiratory samples obtained from individuals suffering from or suspected to suffer from lung cancer.

### SUMMARY

The present invention is based on the observation that analyzing gas samples can be performed when a certain type of ligands capable to chelate with lanthanide(III) ions are used.

According to one aspect the present technology concerns a method for analyzing a gas sample comprising

a) providing one or more receiving solutions;

b) introducing a first gas sample into said one or more receiving solutions;
wherein a lanthanide(III) ion and a ligand, independently of each other, are comprised in said one or more receiving solutions or introduced thereto either before or after introducing said first gas sample;

c) detecting a signal derived from said lanthanide(III) ion in said one or more receiving solutions; and

d) analyzing said first gas sample nonspecifically by comparing said signal with
i) at least one signal derived from said lanthanide(III) ion in said one or more receiving solutions in the presence of said ligand but in the absence of said first gas sample; and/or
ii) at least one signal derived from said lanthanide(III) ion in said one or more receiving solutions in the presence of said ligand, and a second gas sample.

According to another aspect the present technology concerns a kit for analyzing a gas sample, comprising one or more receiving solutions, a ligand, a lanthanide(III) ion, and means for introducing a gas sample into said one or more receiving solutions.

According to another aspect the present technology concerns a computer program product including computer executable instructions for controlling a programmable processor to determine and/or characterize a sample wherein the program is adapted evaluate the data obtainable by a method according to claim 1.

Further objects, aspects, embodiments and details of the present technology are disclosed in dependent claims or will become apparent from the following detailed description and examples.

### BRIEF DESCRIPTION OF THE DRAWINGS

FIG. 1 illustrates of characterization and/or determination of pure and technical grade argons gases in receiving solutions of water (top) and dimethyl sulfoxide (bottom) using six different one or more solutions and the present technology. Y-axis=luminescence signal ratio of pure/technical grade argon gases.

FIG. 2 illustrates an example on characterization and/or determination of breath samples from smoking (3, 4, 7) and non-smoking (1, 2, 5, 6) individuals using the present technology. Y-axis=normalized lanthanide signal.

FIG. 3 illustrates an example on characterization and/or determination of breath samples from a healthy individual (black) and a flu patient (grey) using the receiving solutions including a lanthanide(III) ion, a ligand and modulators and the present technology. Y-axis=normalized lanthanide signal.

FIG. 4 illustrates an example on characterization and/or determination of breath samples from four healthy individuals (dark large filled circles) and lung cancer patients using the present technology. Principle component analysis is presented.

### DETAILED DESCRIPTION OF THE INVENTION

As used herein, the term “analyzing” refers to determining and/or characterizing elements, components, features, or a fingerprint of a sample.

As defined herein the term “gas sample” refers to a sample consisting of any atoms, chemical substances, biomaterial or particles being in gas phase or carried by gas phase. Gas is a state of matter consisting of atoms, chemical substances, biomaterials or particles that have neither a defined volume nor defined shape. Non-limiting examples of gases include air, breath, and smoke. Gases may consist of a single type of atoms, chemical substances, biomaterials or particles or a mixture of them. As used herein, the term “first gas sample” refers to a gas sample to be analyzed by the present method, while the term “second gas sample” refers to a gas sample with which the first gas sample is to be compared. In some embodiments, the second gas sample may be a corresponding sample, a known sample, or any appropriate reference sample.

As defined herein, the term “corresponding sample” refers to a sample that is believed to be virtually identical, highly similar or at least reasonably similar to the first gas sample to be analyzed. This belief of similarity can be due to e.g. origin, i.e. relating to the same or a corresponding process or product, or classification.

As defined herein, the term “known sample” refers to any sample which composition is known in detail or is fully characterized. In some embodiments, a known sample may be employed, for instance, to verify the origin, authenticity, or purity of a first gas sample.

As defined herein, the term “reference sample” refers to any appropriate sample with which the first gas sample is to be compared. A non-limiting example of a reference sample is a breath sample obtained from an healthy individual in cases where the first gas sample is a breath sample obtained from an individual suffering from or suspected to suffer from a disease, such as a lung cancer. In some embodiments, the reference sample is obtained from the same individual as the first gas sample but at a different time point. In such cases, the reference sample may be used to monitor, for instance, progress of a disease or response to a medication.

As defined herein the term “receiving solution” refers to a solution whereto the gas sample is introduced. Suitable receiving solutions include, but are not limited to, aqueous solutions such as water or aqueous buffers, and organic solvents such as dimethyl sulfoxide, dimethyl formamide, ethanol, 1,4-dioxane, acetone, tetrahydrofurane, and any combinations thereof. In some embodiments, a preferred receiving solution is an aqueous solution. However, it is obvious to a person skilled in art that the suitability of a receiving solution may depend on different variables such as the nature of the gas sample to be analyzed and the reactants to be used.

As defined herein the term “array” refers to two or more receiving solutions suitable for analyzing a sample, wherein each solution comprises or is incorporated with a lanthanide(III) ion and a ligand containing 2-5 chelating heteroatoms capable of chelating a lanthanide(III) ion; i.e the ligand is able to form a luminescent lanthanide(III) chelate in the presence of a lanthanide(III) ion.

As defined herein, the term “fingerprint” refers to results obtained through the detection, i.e. measurement of at least one signal obtained from a gas sample in the presence of a lanthanide(III) ion and a suitable chelating ligand. In some embodiments, the fingerprint is obtained through the measurement of the sample in the presence of a plurality of different receiving solutions including one or more lanthanide ions and one or more ligands. If an embodiment of the present technology involves the use of a plurality of identical receiving solutions and the signal derived from the lanthanide ion of more than one type are detected, the fingerprint can include all the results obtained from the identical receiving solutions or alternatively only a representative value, e.g. average, median, mode of the measurements of identical lanthanide(III) chelates or any combination thereof. In some embodiments, at least one signal derived from a lanthanide ion in the presence of a plurality of different ligands may be used to obtain a fingerprint. A fingerprint can further refer to a profile of measured luminescent intensities, lifetimes subjected to numerical processing with an appropriate algorithm and in many preferred alternatives measured luminescent intensities of the chelates of the array are subjected to numerical processing by an appropriate algorithm before comparison with fingerprints of corresponding arrays in the absence of a sample and/or in the presence of a second gas sample.

The present method is a homogeneous method which means that the detection step is not preceded by any physical separation of lanthanide(III) ions bound to chelating ligands or to the sample to be analyzed from non-bound lanthanide(III) ions. This is in contrast to heterogeneous methods which include such a separation.

In the present technology, a ligand and a lanthanide(III) ion, independently of each other, may be added to or included in a receiving solution prior to introducing a gas sample, or added together with a gas sample, or added in a separate addition step before or after introducing the gas sample. As readily understood by a person skilled in the art, the term “independently of each other” means that one of the lanthanide(III) ion and the ligand may be introduced into the receiving solution independently of the other. In other words, administration scheme of the lanthanide(III) ion may be selected independently of the administration scheme of the ligand. Thus, the lanthanide(III) ion and the ligand may be provided into the receiving solution prior to, together with, or after introducing the gas sample, or in any combination thereof.

In some embodiments, instead of employing a lanthanide(III) ion and a ligand as separate entities, they may be employed as a lanthanide chelate. Thus, the present disclosure concerning embodiments, wherein the lanthanide(III) ion and the ligand are provided, added, introduced, or incorporated together by the same administration scheme, applies to embodiments, wherein a corresponding lanthanide chelate is employed, and vice versa.

Thus, a chelating ligand must be used in the present technology or it must be included in a lanthanide chelate to be used in the present technology. The ligand preferably includes an aromatic structure that is able to absorb excitation energy. For an efficient energy transfer the aromatic structure may include heteroatoms such as nitrogen (e.g. pyridine-N), or oxygen (e.g. furan-O) or sulfur (e.g. thiophen-S). The ligand preferably consist (taking the first mentioned chelating heteroatoms into account) 2-5, more preferably 2 or 3 heteroatoms that are able to chelate the lanthanide ion. The heteroatoms may be selected from oxygen (e.g. carboxyl, sulfonate, phosphate, phosphonate, ether, —N═O, C═O), nitrogen (e.g. primary, secondary or tertiary amine or amide), phosphorus (e.g. phosphine or phosphine oxide) and sulfur (e.g. C═S).

It is known in the art that a lanthanide(III) ion is able to form chelates wherein 1-4 two-dentate ligands and up to 3 three-dentate ligands are coordinated to a single lanthanide ion. These types of chelate structures are suitable for the present technology. An example of such a chelate is disclosed in FIG. 3 wherein the receiving solution comprises europium, three ligands (NTA) and one modulator (TOPO).

It is known that β-diketones exhibit keto-enol tautomerism. Accordingly, it is obvious that the present disclosure includes both tautomers although only keto forms are generally presented in figures and formulas.

It is obvious for a person skilled in art that the structure of the luminescent lanthanide chelate used in the present technology varies upon addition of sample solution and other components capable to coordinate with the lanthanide ion and/or interact with each other. Furthermore, the structure of the chelate may be different at different time points. The signal observed in the present technology is derived from a lanthanide ion in the presence of one or more components with or without the sample to be determined and/or characterized.

According to the present technology, the interaction of the sample with the ligand, lanthanide(III) ion, chelate and other components is non-specific. The term non-specific interaction means that the selectivity of the binding or other interaction is not predetermined. The ligand used in the method according to the present technology does not include sample specific recognition elements, such as boronates, germanates or arsenates.

According to one embodiment the sample, the receiving solution, the ligand and the lanthanide(III) ion are mixed in any order. In an exemplary case, the receiving solution comprises the ligand and the lanthanide(III) ion. The gas sample is introduced into the receiving solution including the ligand, and the lanthanide(III) ion. In another example, the gas sample is introduced into the receiving solution and thereafter the ligand and the lanthanide(III) ion are introduced into the receiving solution comprising the gas sample. In a further example, the gas sample is introduced into the receiving solution containing the ligand, and the lanthanide(III) ion is introduced into the solution after introducing the gas sample, or vice versa.

According to an embodiment, the present technology includes a method of analyzing a gas sample including

a) providing one or more receiving solutions;

b) introducing a first gas sample into said one or more receiving solutions;
wherein a lanthanide(III) ion and a ligand, independently of each other, are comprised in said one or more receiving solutions or introduced thereto either before or after introducing said first gas sample;

c) detecting a signal derived from said lanthanide(III) ion in said one or more receiving solutions; and

d) analyzing said first gas sample nonspecifically by comparing said signal with
i) at least one signal derived from said lanthanide(III) ion in said one or more receiving solutions in the presence of said ligand but in the absence of said first gas sample; and/or
ii) at least one signal derived from said lanthanide(III) ion in said one or more receiving solutions in the presence of said ligand, and a second gas sample.

According to an embodiment, the present technology includes a method of analyzing a gas sample, the method including
a) providing one or more receiving solutions;
b) introducing a gas sample into said one or more receiving solutions;
c) introducing a lanthanide(III) ion and a ligand to said one or more receiving solutions comprising said gas sample;
d) detecting a signal derived from the lanthanide(III) ion; and
e) analyzing the gas sample nonspecifically by comparing the signal with
   i) at least one signal derived from said lanthanide(III) ion in said one or more receiving solutions in the presence of said ligand but in the absence of said first gas sample; and/or
   ii) at least one signal derived from said lanthanide(III) ion in said one or more receiving solutions in the presence of said ligand, and a second gas sample.

According to a further embodiment, the present technology includes a method of analyzing a gas sample, including
a) providing one or more receiving solutions including a lanthanide(III) ion and a ligand;
b) introducing a gas sample into said one or more receiving solutions including a lanthanide(III) ion and a ligand;
c) detecting a signal derived from the lanthanide(III) ion; and
d) analyzing the gas sample nonspecifically by comparing the signal with
   i) at least one signal derived from said lanthanide(III) ion in said one or more receiving solutions in the presence of said ligand but in the absence of said first gas sample; and/or
   ii) at least one signal derived from said lanthanide(III) ion in said one or more receiving solutions in the presence of said ligand, and a second gas sample.

According to a still further embodiment, the present technology includes a method of analyzing a gas sample, including

a) providing one or more receiving solutions including one of a lanthanide(III) ion and a ligand;

b) introducing a gas sample into said one or more receiving solutions including the one of the lanthanide(III) ion and the ligand;

c) introducing the other one of the lanthanide(III) ion and the ligand into said one or more receiving solutions including said gas sample and the one of the lanthanide(III) ion and the ligand;

d) detecting a signal derived from the lanthanide(III) ion; and

e) analyzing the gas sample nonspecifically by comparing the signal with
i) at least one signal derived from said lanthanide(III) ion in said one or more receiving solutions in the presence of said ligand but in the absence of said first gas sample; and/or
ii) at least one signal derived from said lanthanide(III) ion in said one or more receiving solutions in the presence of said ligand, and a second gas sample.

According to an exemplary embodiment, the method includes detecting the signal of the lanthanide(III) ion in the absence of the gas sample. Accordingly, the one or more receiving solutions without the sample are detected, preferably at a predetermined time point, for the signal derived from the lanthanide(III) ion in the absence of the sample. The gas sample is then analyzed by comparing the signal derived from the lanthanide(III) ion in the presence of the gas sample and absence of the gas sample.

According to another exemplary embodiment, the method includes detecting the signal of the lanthanide(III) ion with a second gas sample, such as known sample. Accordingly, the one or more receiving solutions including the known sample are detected, preferably at a predetermined time point, for the signal derived from the lanthanide(III) ion. The sample is then analyzed by comparing the signal derived from the lanthanide(III) ion in the presence of the gas sample and the known sample.

According to another exemplary embodiment, the method includes detecting the signal of the lanthanide(III) ion with a corresponding sample. Accordingly, the one or more receiving solutions including the corresponding sample are detected, preferably at a predetermined time point, for the signal derived from the lanthanide(III) ion. The sample is then analyzed by comparing the signal derived from the lanthanide(III) ion in the presence of the gas sample and the corresponding sample.

According to still another exemplary embodiment, the method includes detecting the signal of the lanthanide(III) ion with a reference sample. Accordingly, the one or more receiving solutions including the reference sample are detected, preferably at a predetermined time point, for the signal derived from the lanthanide(III) ion. The sample is then analyzed by comparing the signal derived from the lanthanide(III) ion in the presence of the gas sample and the reference sample.

According to an embodiment the gas sample is analyzed by comparing the signal derived from the lanthanide(III) ion in the presence of the gas sample to be analyzed to one or more of the corresponding signal of the lanthanide(III) ion without a sample, the corresponding signal of the lanthanide(III) ion in the presence of a corresponding sample, the corresponding signal of the lanthanide(III) ion in the presence of a known sample and/or the corresponding signal of the lanthanide(III) ion in the presence of a reference sample.

According to another embodiment the gas sample, one or more receiving solutions without the sample, one or more receiving solutions with the corresponding sample, one or more receiving solutions with the known sample and/or one or more receiving solutions with the reference sample are determined at more than one, preferably predetermined, time point. According to this embodiment the sample can be analyzed based on the signal derived from the lanthanide(III) ion as a function of time.

According to another embodiment, the sample is analyzed based on signal intensity and/or lifetime of the lanthanide(III) signal using time-resolved or gated luminescence measurement. Means and methods for such measurements are well known in the art.

The lanthanide ion of the present technology is selected from europium, terbium, samarium and dysprosium. The preferable lanthanide(III) ions are europium(III) and terbium(III).

According to an embodiment the ligand is a β-diketone of formula (I)

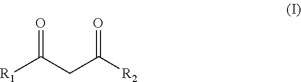

wherein R₁ is an aryl, optionally mono-or multi-substituted, and R₂ is a straight or branched alkyl chain with 1 to 9 carbon atoms substituted with four or more fluorine atoms optionally mono-or multi-substituted with other substituents than fluorine.

According to a preferable embodiment R₁ is selected from the group consisting of phenyl, 9H-fluoren-2-yl, 1-naphthyl, 2-naphtyl, 2-phenanthrolyl, 3-phenanthrolyl, 4-phenanthrolyl, 5-phenanthrolyl, 2-furyl, 3-furyl, 2-benzofuryl, 3-benzofuryl, 2-thienyl, 3-thienyl, 2-pyridyl, 3-pyridyl, 4-pyridyl, 2-thiazolyl, 4-thiazolyl, 5-thiazolyl, 2-benzothiazolyl, 2-benzo [b] thienyl, 3-benzo [b] thienyl, 2-pyrimidyl, 4-pyrimidyl and 5-pyrimidyl.

When R₁ is mono-or multi-substituted each substituent is preferably independently selected from the group consisting of straight or branched alkyl, alkoxy, aryl, aroyl, aryloxy, nitro, amino, cyano, hydroxy, carboxy, chloro, bromo, fluoro and acyl.

According to a preferable embodiment the alkyl chain R₂ is substituted with 3 to 9 fluorine atoms.

Exemplary β-diketones according to present technology are β-diketones of 4,4,4-trifluoro-1-(2-naftyl)-1,3-butanedione (NTA), 2-thienyl-trifluorooacetone (TTA), 4,4,5,5,5-pentafluoro-1-aryl-1,3-pentanedione or 4,4,5,5,6,6,6-heptafluoro-1-aryl-1,3-hexanedione, 1-(2-benzofuryl)-4,4,5,5,5-pentafluoro-1,3-pentanedione, 1-(2-benzofuryl)-4,4,5,5,6,6,6-heptafluoro-1,3-hexanedione, 1-(2-benzo [b] thienyl)-4,4,5,5,5-pentafluoro-1,3-pentanedione and 1-(2-benzo [b] thienyl)-4,4,5,5,6,6,6-heptafluoro-1,3-hexanedione.

According to one preferable embodiment the ligand is selected from TTA and NTA.

According to another embodiment the ligand is a compound of formula (II)

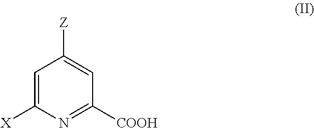

wherein Z is aryl, and X is selected from COOH and CH₂N(CH₂COOH)₂. According to a preferable embodiment the aryl is selected from a group consisting of phenyl, phenylethynyl, napthyl, biphenyl, furan, thiophene, pyridine, pyrazole, imidazole, isothiazole, oxazole, dialkoxyphenyl, and trialkoxyphenyl, preferably trimethoxyphenyl.

According to another embodiment the ligand includes only an aromatic structure that includes two or three chelating heteroatoms. An exemplary ligand according to this embodiment is 1,10-phenantroline (phen).

According to another embodiment the ligand includes two acidic chelating hydroxyl groups. Exemplary ligand according to this embodiment is 2,3-dihydroxynapthalene.

According to another embodiment the ligand is a compound of formula (III)

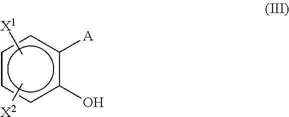

wherein A is OH or COOH, and X¹ and X² are independently selected from halogen, H, and SO₃H. Preferable compounds of formula (III) are fluorosalicylic acid, salicylic acid, and 4,5-dihydrobenzene-1,3-disulfonic acid.

According to another embodiment the ligand is a compound of formula (IV)

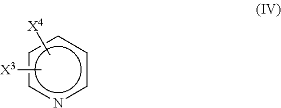

wherein X³ is selected from H, COOH and CONH₂, and X⁴ is selected from COOH and CONH₂. Preferable compounds of formula (IV) are picolinic acid, dipicolinic acid, nicotinic acid and nicotinamide.

According to one embodiment the ligand is selected from the group consisting of dipicolinic acid, picolinic acid, nicotinic acid, nicotinamide, fluorosalisylic acid, 2,3-dihydronaphtalene, salicylic acid, cytosine, chelidamic acid, 4,5-dihydrobenzene-1,3-disulfonic acid, oxilinic acid, ciprofloxacin, 7-amino-1,3-naphhalenesulfonic acid, pyridinedicarboxylic acid, nalidixic acid, and 4-hydroquinoline-2-carboxylic acid.

The receiving solutions may include one or more ligands. An exemplary receiving solution includes NTA and TTA. Also the molar ratio of the ligands may vary from receiving solution to receiving solution and within one receiving solution.

According to a preferable embodiment the receiving solution further includes one or more modulators preferably selected from the group consisting of a synergistic ligand, detergent, protein, peptide, aminocarboxylic acid, carboxylic acid, buffer components and carbohydrate. The function of the modulator in the present technology is preferably to enhance or reduce the signal of the lanthanide chelate and/or assist the formation of individual signal or fingerprint of the sample to be characterized and/or determined.

According to one embodiment the modulator competes with the sample and/or the chelating ligands on the coordination sphere of the lanthanide ion of the chelate and thus modulates the signal derived from the lanthanide(III) ion. The modulator may also enhance the signal e.g. by limiting signal quenching.

According to another embodiment the sample and/or modulator binds to or interacts with the chelating ligand and thus modulates the signal derived from the lanthanide(III) ion.

According to one embodiment one or more of the modulators is a detergent. The detergent is preferably selected from an alkyl aryl polyether alcohol, a zwitterionic compound, and a quaternary ammonium compound. Exemplary detergents are 4-(1,1,3,3-tetramethylbutyl)phenylpolyethyl glycol (Triton X-100), sodium docecyl sulfate (SDS), polyethyleneglycol sorbinan monolaurate (Tween-20), dimethyldodecylphosphine oxide (Apo-12), 3-[(3-cholamidopropyl) dimethylammonio]-1-propanesulfonate and cetyltrimethylammonium bromide (CTAB).

According to another embodiment one or more of the modulators is a synergistic ligand. The synergistic ligand is preferably a Lewis base. A group of such bases consists of N-heterocyclic compounds such as o-phenantroline and of another group consisting of phosphine oxides and phosphines. Exemplary synergistic ligand are trioctylphosphine (TOPO), triphenylphosphine oxide (TPPO), dimethyldecylphosphine oxide (DMDPO), and tributylphosphine oxide (TBPO) and 1,10-phenantroline (phen). The modulators including an aromatic moiety may also act as ligands.

The aminocarboxylic acid is preferably EDTA and DTPA that are known to coordinate metal ions. Exemplary carboxylic acids are citric acid, acetic acid, formic acid, propionic acid and lactic acid.

The method according to the present technology can be performed by using laboratory devices know in art. Exemplary device are microtiter plates, fluidic devices and test tubes.

The method according to the present technology can be performed in aqueous solution such as in water or aqueous buffer(s), in the mixture of water or an aqueous solution and organic solvent(s), and in organic solvent(s). It is obvious for a person skilled in art that the suitability of solvent or solvent system for the method is dependent on the nature of the reagents and reactants used.

According to another embodiment of the present technology an array of at least two different receiving solutions is employed for characterizing and/or determining a sample. It should be understood that in an array, the at least two receiving solutions not mixed but used separately in the method. The separate receiving solutions may be placed e.g. on microtiter plate wells, a fluidic device containing separate reaction wells or separate test tubes.

According to another embodiment a concentration of a sample can be measured.

In order to obtain the fingerprint of the sample, the signal derived from the lanthanide(III) ion of at least two different receiving solutions of the array is detected, preferably at a predetermined, time point in solution. The analyzing of the sample is carried out by comparing the fingerprint of the sample with at least one fingerprint of at least one corresponding sample, at least one fingerprint of an array obtained without a sample, at least one fingerprint of at least one known sample, and/or at least one fingerprint of at least one reference sample.

The signal derived from the lanthanide(III) ion may be used in revealing the fingerprint of an unknown sample by comparing the fingerprint of the signal derived from the lanthanide(III) ion in presence and/or absence of the sample. In comparative studies the fingerprint of the array is compared in presence of samples A, B, C etc. The comparisons of the acquired fingerprints against a library or comparison sets are made by known methods from bioinformatics and data mining. In many embodiments of the present invention fingerprints processed using appropriate algorithms are compared rather than observing the fingerprints of samples as such. Fingerprints and fingerprints processed using appropriate algorithms of the sample can be compared with fingerprints or fingerprints processed, respectively, using appropriate algorithms of:
a sample from the same process taken at an earlier time point,
a sample taken before and/or after adding a substance to the process under study,
a fingerprint of the array in absence of the sample,
a fingerprint of the array in presence of a different concentration of the sample,
a library of fingerprints from known samples,
a library of fingerprints from corresponding samples,
a library of fingerprints related to known anomalities observed at the time points of the library sample,
an algorithm trained with fingerprints of known samples,
an algorithm trained with fingerprints from samples related to known anomalities observed at the time point of the sampling, and or
a fingerprint or set of fingerprints recorded as a function of time.

These algorithms [see e.g. www.dtreg.com and Romesburg C, Cluster Analysis for Researchers, Lulu Press 2004] may e.g. be based on:
neural networks,
independent/principal component analysis,
discriminant analysis,
other clustering algorithms, and
generic expression programming.

In an exemplary embodiment of the present technology the assay is performed as follows:

1) A gas sample is introduced into or through a receiving solution.

2) The receiving solution including the gas sample is allowed to contact two or more solutions including a chelating ligand, a lanthanide(III) ion and modulators.

3) The arrays are read. Reading of results may be carried out with e.g. a luminescence plate reader, a dedicated luminescence array reader, a flow luminometric device, and/or an automated imaging device.

4) The results are interpreted by an appropriate method. The method may involve comparison to a sample from the same process taken at an earlier time point, comparison to a sample taken before and/or after adding a particular substance to the process under study, comparison to a fingerprint of the array in absence of the sample, comparison to a fingerprint of the array in presence of a different concentration of the sample, comparison to a library of fingerprints from known samples, and/or comparison to a library of fingerprints related to known anomalities observed at the time points of the library sample The method may utilize e.g. an algorithm sensitive in differentiation of multidimensional signals, an algorithm trained with fingerprints of known library samples, an algorithm trained with fingerprints from known library samples related to known anomalities observed at the time point of the sampling, and/or comparison to results from the same sample as a function of time after the addition of reaction components. Algorithms of the method may be based on e.g. neural networks, independent component analysis, discriminant analysis and other clustering algorithms and generic expression programming.

In another exemplary embodiment of the present technology the assay is performed as follows:

1) A gas sample is introduced into or through a receiving solution including a lanthanide(III) ion and a ligand.

2) The receiving solution is read. Reading of results may be carried out with e.g. a luminescence plate reader, a dedicated luminescence array reader, a flow luminometric device, and/or an automated imaging device.

3) The results are interpreted by an appropriate method. The method may involve comparison to a sample from the same process taken at an earlier time point, comparison to a sample taken before and/or after adding a particular substance to the process under study, comparison to a fingerprint of the array in absence of the sample, comparison to a fingerprint of the array in presence of a different concentration of the sample, comparison to a library of fingerprints from known samples, and/or comparison to a library of fingerprints related to known anomalities observed at the time points of the library sample The method may utilize e.g. an algorithm sensitive in differentiation of multidimensional signals, an algorithm trained with fingerprints of known library samples, an algorithm trained with fingerprints from known library samples related to known anomalities observed at the time point of the sampling, and/or comparison to results from the same sample as a function of time after the addition of reaction components. Algorithms of the method may be based on e.g. neural networks, independent component analysis, discriminant analysis and other clustering algorithms and generic expression programming.

In another exemplary embodiment of the present technology the assay is performed as follows:

1) A gas sample is introduced into or through a receiving solution including lanthanide(III) ion.

2) The receiving solution including the gas sample is allowed to contact two or more solutions including a chelating ligand and modulators.

3) The arrays are read. Reading of results may be carried out with e.g. a luminescence plate reader, a dedicated luminescence array reader, a flow luminometric device, and/or an automated imaging device.

4) The results are interpreted by an appropriate method. The method may involve comparison to a sample from the same process taken at an earlier time point, comparison to a sample taken before and/or after adding a particular substance to the process under study, comparison to a fingerprint of the array in absence of the sample, comparison to a fingerprint of the array in presence of a different concentration of the sample, comparison to a library of fingerprints from known samples, and/or comparison to a library of fingerprints related to known anomalities observed at the time points of the library sample The method may utilize e.g. an algorithm sensitive in differentiation of multidimensional signals, an algorithm trained with fingerprints of known library samples, an algorithm trained with fingerprints from known library samples related to known anomalities observed at the time point of the sampling, and/or comparison to results from the same sample as a function of time after the addition of reaction components. Algorithms of the method may be based on e.g. neural networks, independent component analysis, discriminant analysis and other clustering algorithms and generic expression programming.

In another exemplary embodiment of the present technology the assay is performed as follows:

1) A gas sample is introduced through a receiving solution including lanthanide(III) ion and modulators.

2) The receiving solution including the gas sample is allowed to contact two or more solutions including a chelating ligand.

3) The arrays are read. Reading of results may be carried out with e.g. a luminescence plate reader, a dedicated luminescence array reader, a flow luminometric device, and/or an automated imaging device.

4) The results are interpreted by an appropriate method. The method may involve comparison to a sample from the same process taken at an earlier time point, comparison to a sample taken before and/or after adding a particular substance to the process under study, comparison to a fingerprint of the array in absence of the sample, comparison to a fingerprint of the array in presence of a different concentration of the sample, comparison to a library of fingerprints from known samples, and/or comparison to a library of fingerprints related to known anomalities observed at the time points of the library sample The method may utilize e.g. an algorithm sensitive in differentiation of multidimensional signals, an algorithm trained with fingerprints of known library samples, an algorithm trained with fingerprints from known library samples related to known anomalities observed at the time point of the sampling, and/or comparison to results from the same sample as a function of time after the addition of reaction components. Algorithms of the method may be based on e.g. neural networks, independent component analysis, discriminant analysis and other clustering algorithms and generic expression programming.

In another exemplary embodiment of the present technology the assay is performed as follows:

1) A gas sample is introduced through a receiving solution including a chelating ligand.

2) The receiving solution including the gas sample is allowed to contact two or more solutions including lanthanide(III) ion and modulators.

3) The arrays are read. Reading of results may be carried out with e.g. a luminescence plate reader, a dedicated luminescence array reader, a flow luminometric device, and/or an automated imaging device.

4) The results are interpreted by an appropriate method. The method may involve comparison to a sample from the same process taken at an earlier time point, comparison to a sample taken before and/or after adding a particular substance to the process under study, comparison to a fingerprint of the array in absence of the sample, comparison to a fingerprint of the array in presence of a different concentration of the sample, comparison to a library of fingerprints from known samples, and/or comparison to a library of fingerprints related to known anomalities observed at the time points of the library sample The method may utilize e.g. an algorithm sensitive in differentiation of multidimensional signals, an algorithm trained with fingerprints of known library samples, an algorithm trained with fingerprints from known library samples related to known anomalities observed at the time point of the sampling, and/or comparison to results from the same sample as a function of time after the addition of reaction components. Algorithms of the method may be based on e.g. neural networks, independent component analysis, discriminant analysis and other clustering algorithms and generic expression programming.

It is clear for a person skilled in the art that the presented protocol and the order of the addition of the reaction components may vary depending e.g. on the application, sample, used reaction and used analysis methods.

According to another embodiment the gas sample dissolves entirely or partially to the receiving solution. It is clear for a person skilled in the art that only some of the gas atoms, chemical compounds, biomaterials and particles is dissolved in the receiving solution and some of the atoms, chemical compounds, biomaterials and particles remains in the gas phase. In a preferred embodiment, the gas sample is partially dissolved to the receiving solution.

According to another embodiment the present technology can be performed in an open or a closed system. In a preferred embodiment the open system is used. In the open system, the majority of the gas sample flows through the receiving solution and is eventually led e.g. to environment or back to the system or process where it was taken. In the open system the pressure change of the receiving solution is not significant compared to the environment while in the closed system the pressure of the receiving solution changes significantly.

In the method according to the present technology a gas sample is introduced into or through a receiving solution. In an exemplary embodiment the gas sample is introduced through a tube to the receiving solution in an open system where non-dissolving gas sample components are led to environment. In another example, the gas sample is by-passed from a process line through the receiving solution and the non-dissolving gas sample components are led back to the process. In a preferred embodiment the gas sample is introduced through the receiving solution including a lanthanide(III) ion and a ligand in an open system.

According to an embodiment the present technology can be used to measure e.g. breath samples to indicate e.g. diseases and disease stages, to distinguish individuals on the basis of their disease status and/or to follow changes in individuals and/or to reveal the content or changes in the content of the gas samples. In a preferred embodiment the method is applied to diagnose lung cancer, to distinguish individuals on the basis of their disease status and/or to follow changes in individuals, and/or to measure changes in the content of the gas sample and impurities of gas sample. In another preferred embodiment, the present technology may be applied to diagnosing and/or monitoring infection diseases, in particular infections of the respiratory system. Non-limiting examples upper respiratory tract infections include common cold, sinusitis, pharyngitis, epiglottitis, and laryngotracheitis, while non-limiting examples of lower respiratory tract infectious include bronchitis, bronchiolitis, pneumonia, and tuberculosis. Alternatively or in addition, the present technology may be applied to diagnosing and/or monitoring any airborne disease, i.e. a disease which is transmitted through the air. In such cases, the pathogen may be a virus, bacterium, or fungus. Non-limiting examples of airborne diseases include avian influenza, and severe acute respiratory syndrome (SARS).

In one aspect, the present invention provides a kit for analyzing a gas sample by the method disclosed herein. The kit comprises one or more receiving solutions provided in separate receptacles, such as reaction wells of a microtiter plate or a fluidic device, or separate vials ar test tubes. In addition, the kit comprises one or more ligands and one or more lanthanide(III) ions in accordance with the above disclosure. Said one or more ligands and one or more lanthanide(III) ions may be provided in said one or more receiving solutions or as separate entities, e.g. in separate vials, to be mixed with said one or more receiving solutions, or in any combinations thereof. Thus, different embodiments of the present technology disclosed above in connection with the present method apply to the present kit as is evident to a person skilled in the art. Additionally, the kit comprises means for introducing a gas sample into said one or more receiving solutions.

According to another aspect, the present technology includes a computer program including software modules for determining information indicative for the sample, in order to evaluate the signal derived from the lanthanide(III) ion under various conditions to obtain fingerprint of the sample; i.e. to characterize and/or determine the sample. The software modules can be e.g. subroutines of functions implemented with a suitable programming language and with a complier suitable for the programming language and the programmable processor.

A computer program product according to an exemplifying embodiment of the present technology includes a computer readable medium e.g. a compact disc, encoded with a computer program according to an embodiment of the present technology.

A signal according to the exemplary embodiment is encoded to carry information defining a computer program according the embodiment.

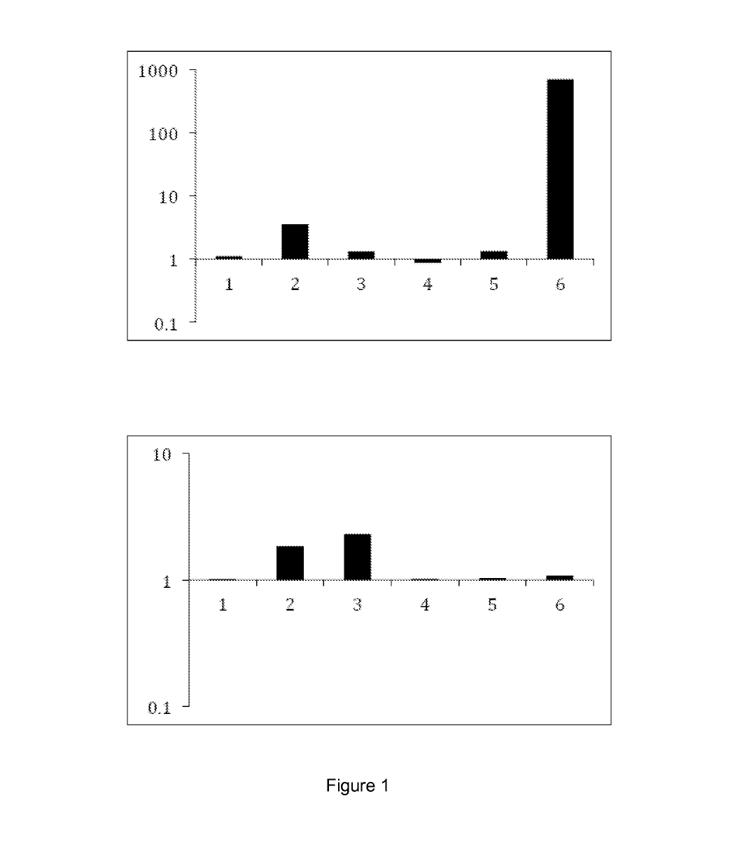

FIG. 1 illustrates characterization of pure and technical grade argon gas (AGA, Turku, Finland) measured in receiving solutions of water (top) or dimethyl sulfoxide (bottom) using the present technology. Six different one or more solutions were used.

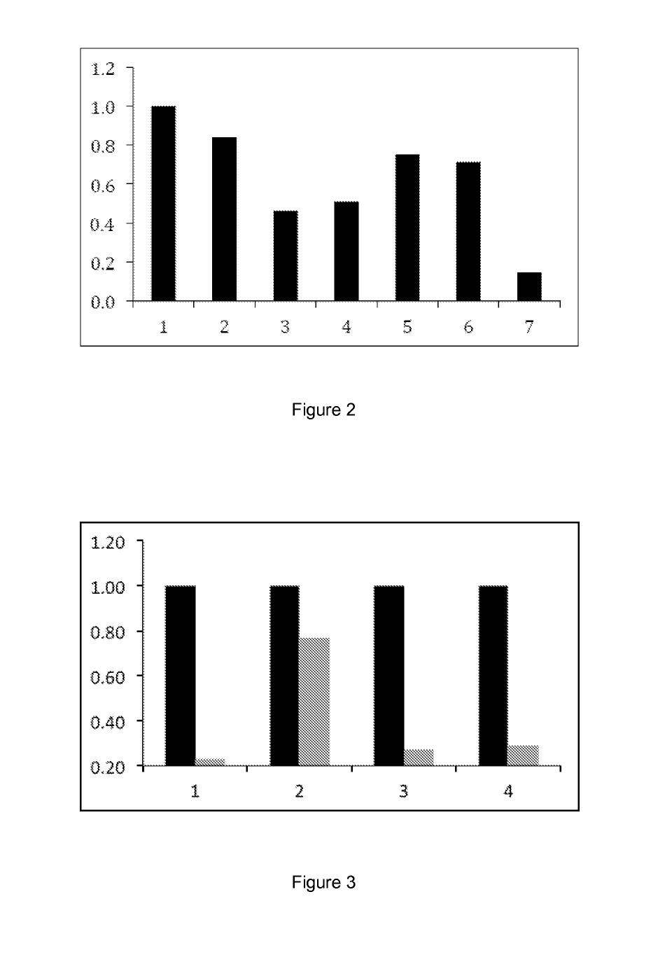

FIG. 2 illustrates determination of seven healthy individuals with three cigarette smokers (individuals 3, 4, 7) and four non-smokers (individuals 1, 2, 5, 6) using the present technology. A single one or more solutions was used.

FIG. 3 illustrates determination of a healthy individual and an individual having a flu using the present technology. Three different receiving solutions including a lanthanide(III), a ligand and different modulators were used.

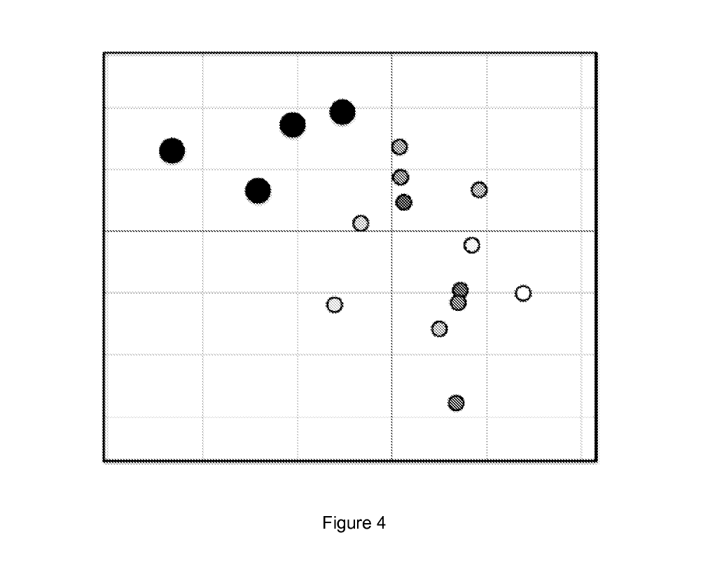

FIG. 4 illustrates determination of four healthy individuals and 12 lung cancer patients using the present technology. Six different one or more solutions were used.

### EXAMPLES

Luminescence emission signal was measured in a gated mode using the Victor² multilabel counter (PerkinElmer, Turku, Finland) and excitation wavelengths of 340-nm for Eu and 320-nm for Tb and emission wavelengths of 615-nm for Eu and 545-nm for Tb.

### Example 1

### Comparative Analysis of Argon Gas Samples

Pure and technical grade argon were introduced through 5 ml of milliQ deionized water (top) or dimethyl sulfoxide (bottom) for three minutes. 100 μL of water or dimethyl sulfoxide were mixed with 10 μL of 30 μM of TbCl₃ and 10 μM of tiron (1), 30 μM of TbCl₃ and 10 μM of nalixid acid (2), 30 μM of TbCl₃ and 10 μM of chelidamic acid (3), 1 μM of EuCl₃, 30 μM of NTA and 30 μM of TOPO (4), 1 μM of EuCl₃, 30 μM of TTA and 30 μM of TOPO (5), 30 μM of TbCl₃ and 10 μM of 4-hydroxy-7-methyl-1,8-naphtyridine-3-carboxylic acid (6) in a 96-well microtiter plate. The luminescence signals of the solutions were measured immediately after 20 minutes incubation. The results are presented in FIG. 2. On the y-axis is shown the signal ratio of pure argon/technical grade argon and on the x-axis is the 6 different chelating solutions. The luminescence signals of the two samples are different in many of the chelating solutions. In case of the chelating solution 6 in water the signal of pure argon gas is approx. 1000-fold higher than that of the technical argon gas. In case of the chelating solution 4 in water the signal of the technical argon gas is higher than the signal of the pure argon.

### Example 2

### Breath Sample Analysis of Cigarette Smokers and Non-Smokers

Individuals exhaled through a tubing to a container containing 10 mL of milliQ deionized water as a receiving solution. One end of the tubing was placed inside the receiving solution and breath sample was collected for 3 minutes. 100 μL of the receiving water solution was mixed with 10 μL of 30 μM of TbCl₃ and 10 μM of tiron in a 96-well microtiter plate. The luminescence signal of the solution was measured. The results are presented in FIG. 2. Normalized luminescence signal is shown on the y-axis. Cigarette smokers (3, 4, 7) and non-smokers (1, 2, 5, 6) are shown on the x-axis. The luminescence signal of the smokers is lower than the signal of the non-smokers indicating that smokers and non-smokers may be distiguished from each other.

### Example 3

### Breath Sample Analysis of a Healthy Individual and a Flu Patient

A healthy individual and a flu patient exhaled through a tubing to a container containing 10 mL of milliQ deionized water and 500 μL of 30 μM of TbCl₃ and 10 μM of Tiron including no modulator (1), or 5 mM of sodium acetate pH 5 (2), 0.0001%-vol of Triton X-405 (3) and 10%-vol of DMSO (4) as modulators. One end of the tubing was placed inside the receiving solution and breath sample was collected for 3 minutes. 100 μL of the receiving water solution including the sample and the lanthanide(III) ion and the ligand was added to a 96-well microtiter plate. The luminescence signals of the solutions were measured. The results are presented in FIG. 3. Normalized luminescence signal is shown on the y-axis. The healthy individual (black) and the flu patient can be distinguished based on the measured signals as the signal levels of the flu patient are lower.

### Example 4

### Breath Sample Analysis of Healthy Individuals and Lung Cancer Patients

Individuals exhaled through a tubing to a container containing 10 mL of milliQ deionized water as a receiving solution. One end of the tubing was placed inside the receiving solution and breath sample was collected for 3 minutes. 100 μL of the receiving water solution was mixed with 10 μL of 30 μM of TbCl₃ and 10 μM of tiron (1), 30 μM of TbCl₃ and 10 μM of nalixid acid (2), 30 μM of TbCl₃ and 10 μM of chelidamic acid (3), 1 μM of EuCl₃, 30 μM of NTA and 30 μM of TOPO (4), 1 μM of EuCl₃, 30 μM of TTA and 30 μM of TOPO (5), 30 μM of TbCl₃ and 10 μM of 4-hydroxy-7-methyl-1,8-naphtyridine-3-carboxylic acid (6) in a 96-well microtiter plate. The luminescence signal of the solution was measured. The results are presented in FIG. 4 using a principle component analysis. On the y-axis is shown the first principle component and on the x-axis is shown the second principle component. The lung cancer patients (small circles with different grey colors) can be distinguished from the healthy individuals (large black circles).

The specific examples provided in the description given above should not be construed as limiting the scope and/or applicability of the appended claims.

1. A method of analyzing a gas sample, the method comprising
a) providing one or more receiving solutions;
b) introducing a first gas sample into said one or more receiving solutions;
wherein a lanthanide(III) ion and a ligand, independently from each other, are comprised in said one or more receiving solutions or introduced thereto either before or after introducing said first gas sample;
c) detecting a signal derived from said lanthanide(III) ion in said one or more receiving solutions; and
d) analyzing said first gas sample nonspecifically by comparing said signal with
i) at least one signal derived from said lanthanide(III) ion in said one or more receiving solutions in the presence of said ligand but in the absence of said first gas sample; and/or
ii) at least one signal derived from said lanthanide(III) ion in said one or more receiving solutions in the presence of said ligand, and a second gas sample.

2. The method according to claim 1 wherein said ligand comprises 2-5 chelating heteroatoms.

3. The method according to any of claims 1-2 wherein said ligand comprises an aromatic structure.

4. The method according to any of claims 1-3 wherein said ligand is selected from the group consisting of a β-diketone of formula (I) 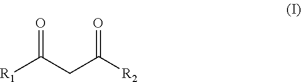
wherein R₁ is aryl, optionally mono- or multisubstituted, R₂ is a straight or branched alkyl chain with 1 to 9 carbon atoms substituted with three of more fluorine atoms optionally mono-or multisubstitued with other substituents other than fluorine, a compound of formula (II) 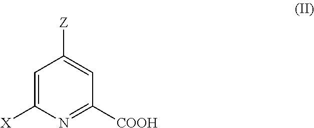
wherein Z is H or aryl, and X is selected from COOH and CH₂N(CH₂COOH)₂, and 1,10-phenantroline (phen).

5. The method according to any of claims 1-3 wherein the ligand is selected from a compound of formula (III) 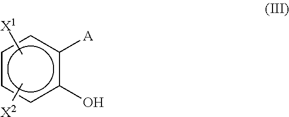
wherein A is OH or COOH, and X¹ and X² are independently selected from halogen, H, and SO₃H.

6. The method according to claim 5 wherein the ligand is selected from fluorosalicylic acid, salicylic acid, and 4,5-dihydrobenzene-1,3-disulfonic acid.

7. The method according to any of claims 1-3 wherein the ligand is selected from a compound of formula (IV) 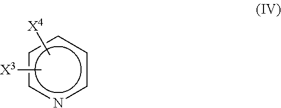
wherein X³ is selected from H, COOH and CONH₂, and X⁴ is selected from COOH and CONH₂.

8. The method according to claim 7 wherein the ligand is selected from picolinic acid, dipicolinic acid, nicotinic acid and nicotinamide.

9. The method according to any of claims 1-3 wherein the ligand is selected from the group consisting of dipicolinic acid, picolinic acid, nicotinic acid, nicotinamide, fluorosalisylic acid, 2,3-dihydronaphtalene, salicylc acid, cytosine, chelidamic acid, 4,5-dihydrobenzene-1,3-disulfonic acid, oxilinic acid, ciprofloxacin, 7-amino-1,3-naphhalenesulfonic acid, pyridinedicarboxylic acid, nalidixic acid, and 4-hydroquinoline-2-carboxylic acid.

10. The method according to any of claims 1-9 wherein said lanthanide is selected from europium, terbium, samarium and dysprosium, preferably europium or terbium.

11. The method of claim any of claims 1-10 wherein said one or more solutions and/or said one or more receiving solutions further comprises one or more modulators selected from the group consisting of synergistic ligand, detergent, protein, peptide, aminocarboxylic acid, carboxylic acid, buffer components, and carbohydrate.

12. The method of claim 11 wherein the synergistic ligand is selected from the group consisting of phosphine oxide preferably trioctylphosphine (TOPO), triphenylphosphine oxide (TPPO), dimethyldecylphosphine oxide (DMDPO), and tributylphosphine oxide (TBPO) and 1,10-phenantroline (phen).

13. The method of claim 11 or 12 wherein said detergent is selected from 4-(1,1,3,3-tetramethylbutyl)phenylpolyehyl glycol (Triton X-100), hexadecyltrimethylammonium bromide (CTAB), sodium docecyl sulfate (SDS), polyethyleneglycol sorbinan monolaurate (Tween-20) and dimethyldodecylphosphine oxide (Apo-12).

14. The method according to claim 1 wherein said signal derived from said lanthanide(III) ion is detected at a predetermined time point.

15. The method according to any of the claims 1-14 wherein said method is applied to analyze a breath sample for the presence or absence of a disease, preferably lung cancer, or for determining a disease stage, for distinguishing individuals on the basis of their disease status, or for monitoring changes in a disease status, or for monitoring the content or changes in the content of the gas sample, preferably impurities of the gas sample.

16. A kit for analyzing a gas sample according one of claims 1-15, comprising one or more receiving solutions, a lanthanide(III) ion, and a ligand and means for introducing a gas sample into said one or more receiving solutions.

17. The kit according to claim 16 wherein said ligand comprises 2-5 chelating heteroatoms.

18. The kit according to any of claims 16-17 wherein said ligand comprises an aromatic structure.

19. The kit according to any of claims 16-18 wherein said ligand is selected from a group consisting of a β-diketone of formula (I) 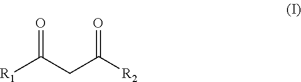
wherein R₁ is aryl, optionally mono- or multisubstituted, R₂ is a straight or branched alkyl chain with 1 to 9 carbon atoms substituted with three of more fluorine atoms optionally mono-or multisubstitued with other substituents other than fluorine, a compound of formula (II) 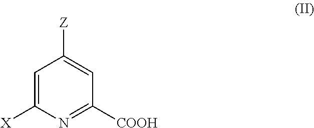
wherein Z is aryl, and X is selected from COOH and CH₂N(CH₂COOH)₂ and 1,10-phenantroline (phen).

20. The kit according to any of claims 16-18 wherein the ligand is selected from a compound of formula (III) 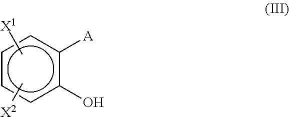
wherein A is OH or COOH, and X¹ and X² are independently selected from halogen, H, and SO₃H.

21. The kit according to claim 20 wherein the ligand is selected from fluorosalicylic acid, salicylic acid, and 4,5-dihydrobenzene-1,3-disulfonic acid.

22. The array according to any of claims 16-18 wherein the ligand is selected from a compound of formula (IV) 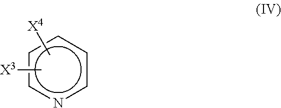
wherein X³ is selected from H, COOH and CONH₂, and X⁴ is selected from COOH and CONH₂.

23. The kit according to claim 22 wherein the ligand is selected from picolinic acid, dipicolinic acid, nicotinic acid and nicotinamide.

24. The kit according to any of claims 16-18 wherein the ligand is selected from the group consisting of dipicolinic acid, picolinic acid, nicotinic acid, nicotinamide, fluorosalicylic acid, 2,3-dihydronaphtalene, salicylcic acid, cytosine, chelidamic acid, 4,5-dihydrobenzene-1,3-disulfonic acid or its disodium salt, oxilinic acid, ciprofloxacin, 7-amino-1,3-naphhalenesulfonic acid, pyridinedicarboxylic acid, nalidixic acid, and 4-hydroquinoline-2-carboxylic acid.

25. The kit according to any of claims 16-24 comprising a lanthanide(III) ion selected from europium, terbium, samarium and dysprosium.

26. The kit according to claim 25 wherein the lanthanide is europium or terbium.

27. The kit according any of claims 16-26 wherein further comprising one or more modulators selected from the group consisting of synergistic ligand, detergent, protein, peptide, aminocarboxylic acid, and carbohydrate.

28. The kit according to claim 27 wherein the synergistic ligand is selected from the group consisting of phosphine oxide preferably trioctylphosphine (TOPO), triphenylphosphine oxide (TPPO), dimethyldecylphosphine oxide (DMDPO), and tributylphosphine oxide (TBPO) and 1,10-phenantroline (phen).

29. The kit or claim 27 or 28 wherein said detergent is selected from 4-(1,1,3,3-tetramethylbutyl)phenylpolyehyl glycol (Triton X-100), sodium docecyl sulfate (SDS), Polyoxyethylene (20) sorbitan monolaurate (Tween-20), dimethyldodecylphosphine oxide (Apo-12), 3-[(3-cholamidopropyl) dimethylammonio]-1-propanesulfonate and cetyltrimethylammonium bromide (CTAB).

30. The kit according to claim 16 wherein said signal derived from said lanthanide(III) ion is detected at a predetermined time point.

31. The kit according to any of the claims 16-30 wherein said method is applied to analyze a breath sample for the presence or absence of a disease, preferably lung cancer, for determining a disease stage, for distinguishing individuals on the basis of their disease status, or for monitoring changes in a disease status, or for monitoring the content or changes in the content of the gas sample, preferably impurities of the gas sample.

32. A computer program product including computer executable instructions for controlling a programmable processor to analyze a gas sample wherein the program is adapted to evaluate the data obtainable by a method according to any of claims 1-31.

33. A computer program product comprising computer readable medium encoded with a computer program according to claim 32.
